(19) United States
(12) Patent Application Publication
(10) Pub. No.: US 2004/0199212 A1
(43) Pub. Date: Oct. 7, 2004

(54) External patient alerting system for implantable devices

(76) Inventors: David R. Fischell, Fair Haven, NJ (US); Robert E. Fischell, Dayton, MD (US); Jonathan Harwood, Rumson, NJ (US)

Correspondence Address: Robert E. Fischell, Sc.D., 14600 Viburnum Drive, Dayton, MD, 21036, US

(21) Appl. No.: 10/403,210

(22) Filed: Apr. 1, 2003

(51) Int. Cl. A61N 1/18

(52) U.S. Cl. 607/32

(57) ABSTRACT

Disclosed is a patient alerting device (PAD) that can be positioned close enough for effective near-field communication with an implanted medical device such as a pacemaker or implantable cardiac defibrillator. The PAD is designed to warn the patient when a specific event is detected by the implanted device. The alarm mechanism could be a sound, a vibration and/or a visible display. A communications repeater can be used with the PAD to allow the implanted device to communicate via the PAD with remotely located external equipment through proprietary or standardized means. Means to initiate communication with the implanted device include a magnet within the PAD that triggers a magnetic switch inside the implanted device or by a button which can be depressed to inform the implanted device that the PAD is active and close enough to begin communication. The PAD could also include a button to shut off an alarm that is in the PAD. The PAD can also include means to provide battery status to the patient for both the battery in the implanted device and in the PAD. The significance of the basic alarm PAD is its ability to provide continuous patient alarm capabilities for already implanted devices as well as future implants which have only near-field communication.

### FIELD OF USE

This invention is in the field of systems combining implantable medical devices with externally located equipment with which the implanted device communicates.

### BACKGROUND OF THE INVENTION

Over the last 50 years there has been a significant increase in the variety of implantable electronic medical devices for both diagnostic and therapeutic purposes. These devices include cardiac pacemakers (pacers), implantable cardiac defibrillators (ICDs), implantable drug pumps, implantable neurostimulators for pain and disorders of the brain, bone growth stimulators implantable cardiac loop recorders and cochlear implants The latest technologies for such implantable electronic devices include a wide range of event detection capabilities. The majority of such systems use event detection to trigger the delivery of therapy and/or initiation of data recording for later analysis by medical practitioners. Programming and communication with almost all of these implantable devices require placement of an electromagnetic coil (essentially an antenna) within a very short distance (typically less than an inch) of the implant's subcutaneous location. As a result of this “near-field” telemetry, communication of the implant to external equipment has typically required a relatively large wired wand that is placed in close proximity to the device. The wand is connected by electrical conductors to the external equipment with which the implant communicates. Such external equipment has typically included device programmers and home data transmission systems for sending data collected by the implant to a remotely located database over phone lines. Recently one company (Biotronik) has introduced a pacemaker with relatively long-range telemetry (approximately 3 meters) that provides data transmission to an external receiving system. While this type of telecommunication (long-range telemetry) can be implemented in future generations of the various implantable devices listed above, there is no present means to continuously monitor patients who now have implanted devices that have only near-field telemetry capabilities.

Many of the existing near-field implantable devices have the capability for feature enhancements through software upgrades that could provide a multitude of valuable and potentially life saving capabilities if a means of alerting the patient existed that could function with the limitations of near-field communication. Fischell et al in U.S. Pat. Nos. 6,112,116 and 6,272,379, that are incorporated herein by reference describe drug pumps, pacers and ICDs that can detect cardiac events like acute myocardial infarction (AMI). Such a detection capability if added to an already implanted pacer or ICD that has only near-field telemetry capability could be life saving if a means existed to alert the patient.

### SUMMARY OF THE INVENTION

The present invention is a patient alerting device (PAD) that can be positioned close enough for effective near-field communication with an implanted medical device such as a pacer or ICD. Positioning the PAD in close proximity to the implanted device could be accomplished by the use of an adhesive patch that attaches the PAD to the patient's skin, a special vest, a shirt with a pocket where the PAD can be placed or attached with Velcro or a clip for attachment to regular clothing such as underwear. Patient alerting devices (PADs) could be produced in a number of different configurations with features including:

1. A basic patient alarm device that can warn the patient when a specific event is detected by the implanted device. The alarm mechanism could be a sound, a vibration and/or a visible display.

2. The PAD may be worn directly over the implant for continual monitoring or it may be placed within the telemetry range of the implant when the patient experiences one or more symptoms related to a medical event.

3. A communications repeater that can allow the implanted device to communicate with remotely located external equipment through proprietary or standardized means. Standardized means include wireless transmission standards such as Bluetooth, 802.11a, b or g, GPRS, CDMA, TDMA, GSM, SMS and other paging and cell phone standards.

4. Means to initiate communication with the implanted device. This could be by a magnet within the PAD that triggers a magnetic switch inside the implanted device or by a button which can be depressed to inform the implanted device that the PAD is active and close enough to begin communication. Alternately, it is envisioned that PAD might transmit once or more times a minute an “are you there” message that would allow it to automatically enable communication with the implant when it is within near-field communication range.

5. A button to shut off an alarm that is in the PAD.

6. Means to provide battery status to the patient for both the battery in the implanted device and in the PAD.

The physical structure of the patient alerting device (PAD) would typically be a small plastic case that contains a battery, a push button and the PAD electronics that is worn over the subcutaneous location of the implanted device. An adhesive patch could be placed on the patient's skin over the pacer implant and the patient alerting device could be attached to that adhesive patch. Once in place, the button on the PAD would be depressed to initiate any one or all of the following actions:

1. Check the battery in the PAD (a spoken message, beep or buzz, sound or vibration or a visible display could be used to confirm “Battery OK”).

2. Initiate communication with the implant. This could be asynchronous communication or, to reduce battery use, the initial communication could synchronize the two devices so that the PAD need only look at a preset interval for communication from the implant allowing longer life and much smaller size for the PAD. For example, after initial synchronization, the PAD might wake up only to look for messages from the implant for 100 msec every 30 seconds. This very reduced duty cycle would greatly enhance the PAD's battery life.

3. Enable the implant to initiate event detection algorithms that can result in an alarm if a cardiac event is detected.

4. A device communication alert mode if the PAD loses communication with the implant.

5. Enable the basic alarm PAD to be ready to alert the patient with a spoken message, a sound, a vibrations an electric shock and/or a visible display when an appropriate alarm message is received from the implant.

It is also envisioned that the implant might already have detected an adverse event, and when the PAD and implant initiate communications, the alarm in the PAD could be turned on immediately. An alternate embodiment of the basic alarm PAD could include a magnet within the PAD rather than a button to initiate communication by activation of a magnetic switch within the implant.

A vest or special piece of clothing with means for holding or attaching the PAD could be a more comfortable system for long-term use of the PAD as compared with adhesive tape attachment. For example, the PAD could be securely placed over the implanted device by means of a clip that can attach the PAD to a tee shirt or an undergarment such as a woman's bra. Alternately the patient could wear a specially designed vest that uses Velcro or a pocket to position the PAD over the site of the implanted pacer or ICD.

A first embodiment of such a PAD, a basic alarm PAD, would alert the patient in the event of a detected medical event by an audible or vibratory alarm, a battery, and a single push button. As one example, the basic alarm PAD could be used with an implanted pacer that has cardiac event detection capability such as described in the previously referenced Fischell et al patents.

The significance of the basic alarm PAD is its ability to provide patient alarm capabilities for already implanted devices as well as future implants which have only near-field communication. Near-field communication has the advantage of requiring much less power than long-range telemetry allowing the implant to be smaller and/or last longer than an equivalent device that uses long-range telemetry.

The basic alarm PAD could also have a connector allowing attachment of a long-range communication module that would enable the basic alarm PAD to send and receive information to and from remotely located external equipment. Alternately, long-range communication capability to remotely located external equipment could be integral to the PAD unit.

Such a PAD having long-range communication capability is a second important embodiment of the PAD, allowing the PAD to retransmit data from the implant to remotely located external equipment and services. This second embodiment a repeater PAD, allows near-field implants to communicate with the outside world allowing a wide range of important benefits including:

1. Remote monitoring of patient status through data downloads of ECG, EEG, blood pressure, blood glucose level, etc.

2. Remote diagnosis of patient adverse health events such as heart attacks and epileptic seizures.

3. Summoning help in the event of an adverse health event.

The repeater PAD might use limited-range telemetry capability (e.g., within the patient's home at a range of 3 to 100 meters) to send patient information to local equipment such as a cell phone, a Pocket PC or Palm PDA or a wired modem for sending information over existing wired voice or data networks Bluetooth wireless telemetry is now available to computers, cell phones, PDAs, printers and other electronic devices and could be an ideal means for the repeater PAD to transmit and receive data. For example, a Bluetooth enabled tablet PC or printer at a doctor's office or emergency room could quickly print or display important patient data for medical diagnosis.

Rather than limited-range telemetry only within the patient's home to another device that can transmit data over long distances (e.g. modems, cell phones etc.) the repeater PAD itself might contain the means for long-distance data communication. This would require integration of long-distance wireless communication circuitry with an appropriate antenna. It is most likely that a protocol such as GPRS, CDMA, TDMA, GSM, SMS or other paging and cell phone standards would be used by the limited-range repeater PAD.

Either through a second device or using integrated long-distance communication means, one can envision the repeater PAD immediately placing a call out to a patient alarm monitoring service much like a burglar alarm service. This would be of particular importance in connection with the detection of a life threatening events such as an acute myocardial infarction (AMI) by the implant.

The alarm monitoring service could then confirm the detection of a cardiac event and summon an ambulance to get the patient quickly to a hospital for treatment. Upon arrival at the hospital the repeater PAD would allow the patient's stored and real time electrogram information to be displayed and/or printed to facilitate the fastest possible treatment for AMI. It is of course clearly understood that the shortest possible time to treatment after an AMI is most important in preventing death and damage to the patient's myocardial tissue.

Another example of use of the repeater PAD is for ICDs where an ambulance might be summoned directly when the device detects ventricular fibrillation. Additional messages to the patient's cardiologist could also alert the doctor that the patient had a cardiac event. The calling out might be by direct communication through cellular telephone data or by two-way paging capabilities or it Ought require a Bluetooth enabled cell phone that would act as a second repeater and would retransmit the data sent from the repeater PAD out to a remote system or service. Fischell et al in U.S. patent application Ser. No. 10/251,505 (which is included herein by reference) describe a comprehensive rescue service designed for fast response to an AMI.

A repeater PAD could be used to retransmit diagnostic data to a remote location without any specific patient alerting signals or it might retransmit diagnostic data only as the result of a detected event.

A third embodiment of the PAD (a memory PAD) would include integrated data storage (e.g. flash memory) and/or a standardized slot for insertion of removable flash memory cards. Such cards include the current standardized flash memory cards, like compact flash cards, smart media cards, memory sticks, PCMCIA memory cards and microdrives, secure digital (SD) cards and USB connector flash memory devices. This would provide a number of important capabilities and enhancements to existing and future implanted devices. For example, the Medronic REVEAL implantable loop recorder has a 72 hour ecg storage to help find anomalous heart signal events. The memory PAD would allow existing pacers and ICDs to have loop recording capabilities and could, with enough data storage or using multiple removable flash memory cards provide patient monitoring over much longer periods. The memory PAD could be used in conjunction with the REVEAL to extend the loop recording to weeks or months. Data storage by the memory PAD could be continuous patient initiated or triggered by an event detected by the implant.

It is also envisioned that event detection might be performed by the PAD itself on diagnostic data received from the implant rather than having event detection performed by the implant itself. This capability, could be integrated into any of the PAD embodiments described above. For example, a memory PAD might be acting as a cardiac Holter monitor or loop recorder receiving continuous or periodic electrogram data from an implanted pacemaker. ST shift detection such as that described by Fischell et al in U.S. patent application Ser. No. 10/251,505 could be performed by the PAD and the detection of an event such as acute myocardial infarction (i.e., a heart attack) could result in an action that could include any or all of the following:

1. Alerting the patient using the basic alarm PAD functionality.

2. Transmitting the electrogram and alarm data to a remote diagnositic center for review by a medical professional using repeater PAD functionality.

3. Storing the electrogram and alarm data using memory PAD functionality.

4. Commanding an implanted medical device, for example a drug pump, to provide a specific therapy to the patient.

It should be clearly understood that combinations of the basic alarm PAD, the repeater PAD and the memory PAD are desirable. A PAD having all three capabilities would be the most desirable as it could used as a basic alarm PAD, a repeater PAD, a memory PAD or any combination.

Thus it is an object of this invention to have a basic alarm PAD, repeater PAD or memory PAD that can be worn by the patient to alert the patient with an alarm signal when a detected event is detected by an implanted medical device. The alarm signal may be a speech segment, a sound, a vibration, an electric shock or a visual display. The detected event can be any medical condition that can be sensed by the implanted device which includes (but is not limited to) bradycardia, ventricular fibrillation, cardiac arrhythmia, acute myocardial infarction, coronary artery ischemia, either too high or too low blood glucose, either too high or too low blood pressure, a pre-cursor of an epileptic seizure, the aura of a migraine headache, or any other medical condition that the implanted medical device can detect.

Another object of the present invention is to have the basic or repeater alarm PAD having means to turn off a patient alarm signal.

Still another object of the present invention is to have means to notify the implant that the PAD is in place and ready for communication. Such notification could be automatic via electronics or by use of magnet or manually initiated after the depression of a button (or switch) on the PAD.

Still another object of the present invention basic alarm PAD is the means (such as a vest, clip onto a bra, etc.) for placing and maintaining the PAD within the near-field communication range of the implanted medical device.

Still another object of the present invention is to have a repeater PAD that acts as a repeater to allow a near-field capable implanted medical device to gain the capability of medium and long-range data communication.

Still another object of the present invention is to have the repeater PAD provide telemetry from the implant to nearby external equipment with a standardized protocol such as Bluetooth or 802.11b.

Still another object of the present invention is to have the repeater PAD provide long-range data communication directly to and from remotely located external systems and services with a standardized protocol such as CDMA, TDMA, GPRS or SMS.

Still another object of the present invention is to have the repeater PAD provide data communication to and from telecommunications devices using a standardized protocol such as Bluetooth or 802.11b, the telecommunications devices having the capability to provide long-range data communication to and from remotely located external systems and services with a standardized protocol such as CDMA, TDMA, GPRS or SMS.

Still another object of this invention is to have an implanted defibrillator or pacemaker that can sense a cardiac adverse event and trigger the alerting of the patient by a PAD worn by the patient directly over the implant.

Yet another object of this invention is to have the PAD itself capable of detecting an event from diagnostic data received from an implanted medical device.

These and other objects and advantages of this invention will become obvious to a person of ordinary skill in this art upon reading of the detailed description of this invention including the associated drawings as presented herein.

### BRIEF DESCRIPTION OF THE DRAWINGS

FIG. 1 illustrates the configuration for use of the present invention PAD in conjunction with a subcutaneously implanted medical device.

FIG. 2 illustrates the configuration of a two-piece repeater PAD.

FIG. 3 is a block diagram showing the elements within an implanted device and within the basic alarm PAD.

FIG. 4 is a block diagram of the elements within a repeater PAD.

FIG. 5 is a block diagram of the elements within a basic alarm PAD with additional patient monitoring flash memory capability.

FIG. 6 illustrates the physical configuration of a memory PAD with a flash memory slot.

FIG. 7 is a block diagram of the elements within a combination PAD combining the capabilities of a basic alarm PAD, a repeater PAD and a memory PAD with additional event detection capability.

### DETAILED DESCRIPTION OF THE DRAWINGS

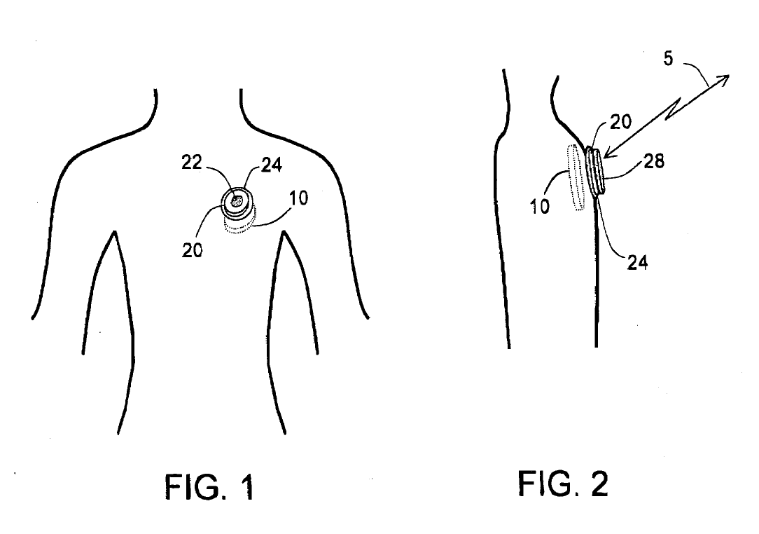

FIG. 1 illustrates the basic configuration of the present invention PAD 20 in conjunction with a subcutaneously implanted medical device 10. The purpose of the basic alarm PAD 20 is to provide means to alert the patient to a condition detected by the implant 10 through an alerting signal generated by the PAD that might be a sound, a vibration (like a cell phone or pager) or a visual display. The medical device 10 can be a pacemaker, an implantable cardiac defibrillator (ICD), a tissue stimulator for pain or bone growth, a neurostimulator, an implantable diagnotsic device such as the REVEAL by Medtronic, an implantable drug pump or any combination of these types of implantable medical devices. Today almost all implantable medical devices 10 use a magnetic induction coil to provide short-range data communication with external equipment. The basic alarm PAD 20 is attached to the patient's skin with the adhesive pad 24 and has one control button 22 that may be programmed to perform several different functions. For example, when the PAD is situated at the appropriate site above the implanted device 10, the button 22 could be depressed to initiate communication with the implant 10 enabling software within the implant 10 to know that the PAD is in place and available for patient alarming. The adhesive pad 24 could have a mechanical or adhesive attachment to the PAD.

An alternative embodiment to initiate communication between the implant and the PAD could be the inclusion of a permanent magnet in the PAD 20 that would actuate a magnetic switch in the implant 10 so that it is only necessary to properly place the PAD 20 in close proximity to the implant to enable communication. The button 22 may also serve as a means to check that both implant 10 and PAD 20 are alive and functioning properly by having the PAD 20 respond with a sound or vibrational signal when the button 22 is pushed. Finally, the button 22 can be programmed to turn off a patient alerting (alarm) signal. Also, once the communication link with the implant 10 is activated, the PAD 20 will alert the patient if the ink is lost due to the PAD 20 coming off or being moved away from its site over the implant 10.

It is also envisioned that a PAD with no button could simply produce the alarm signal for a specific time duration after an event is detected by the implant or that to get the alarm to turn off, the PAD 20 could be removed from its site over the implant 10. The basic alarm PAD 20 might be a low cost disposable unit that could have a life from 2 weeks to several months after which a new PAD 20 would be placed over the implant. Alternatively, the PAD could be rechargeable or have replaceable batteries.

FIG. 2 illustrates the configuration of the repeater PAD which is a basic alarm PAD 20 with an attached repeater module 2X. The repeater module 28 would connect to the electronics within the PAD 20 to facilitate long-range data communications via RF signals 5 with remotely located external equipment (not shown). In this way the PAD 20 with repeater module 28 can allow the implant to transmit alarms and diagnostic data to the patient's doctor or to a central monitoring service that could respond to patient alarms much like a burglar alarm service would respond to alarms generated by a home burglar/fire alarm system. Such a comprehensive rescue/monitoring service is described by Fischell et al in U.S. patent application Ser. No. 10/051,743 which is included herein by reference. This cited Fischell et al invention envisions implanted devices with fairly long-range telemetry that are designed to work with an external alarm system that can perform the same function as the PAD 20 with repeater module 28. The difference is that in this cited Fischell et al invention, the implant was specifically designed for this purpose. On the other hand, the present invention will allow advanced capabilities for existing implantable devices 10 with minor (or no) software changes and (of course) no hardware changes. For example, instead of needing a wand to be placed over the site of an implanted pacer, the repeater PAD system could be used to transmit data to a remotely located monitoring system with the patient lying in a hospital bed.

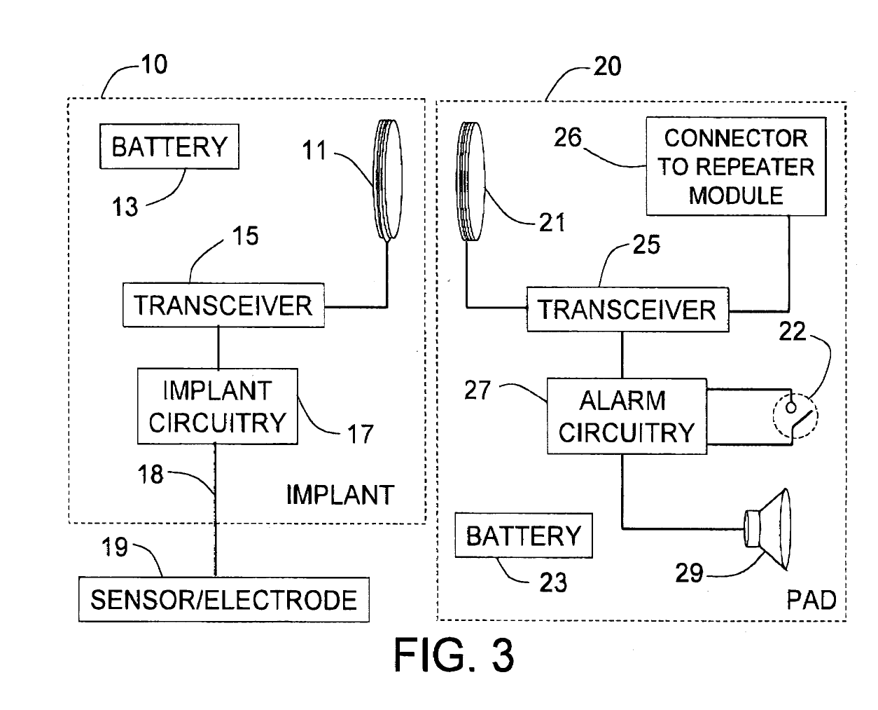

FIG. 3 is a block diagram of the elements within an the implanted device 10 and a basic alarm PAD 20. The implanted device 10 comprises a battery 13, implant (e.g., pacer) circuitry 17 that provides the functionality of the implant 10 when associated with sensors and or electrodes 19. For example, in a pacemaker, the electrodes of the lead would connect to pacing and heart signal sensing circuitry. A transceiver 15 connected to a transmit/receive coil 11 provides the means for short-range data communication between the implant 10 and the PAD 20. The PAD 20 has a transmit/receive coil 21 connected to the transceiver 25. Alarm circuitry 27 connected to the speaker 29 can process incoming signals from the implant 10 and generate alarm or alerting signals to be emitted by the speaker 29. The button (or a switch) 22 can turn off the alarm or alerting signal and can be used as described with FIG. 1 to initiate data communication between the implant 10 and the PAD 20 or to check the status of either or both devices. A connector to a repeater module 26 allows connection of an attachable repeater module 28 as shown in FIG. 2

The typical method of use of the basic alarm PAD 20 without the repeater module 28 is as follows:

1. Connect the PAD 20 to the adhesive pad 24 or place the PAD in a specially designed vest, etc.

2. Expose the adhesive backing on the adhesive pad 24 by removing the protective plastic covering.

3. Attach the adhesive pad 24 to the skin above the site of the implanted device 10.

4. Depress the button 22 to initiate data communication between the PAD 20 and the implant 10.

5. A sound will be emitted from the speaker 29 indicating that the connection is made and that the battery status of both devices is “OK”. If no sound occurs, the PAD battery 13 is low and a new PAD 20 should be opened and used or the PAD should be recharged. A different sound might be used for a battery check of the PAD 20 before it is place in the near-field communication range of the implanted device 10.

6. If a condition requiring a patient alert or alarm occurs and is detected by the implant 10, then a message will be sent by the implant's transceiver 15 through the coil 11 to the coil 21 and transceiver 25 of the PAD 20. The alarm circuitry 27 will interpret the signal and cause the appropriate alarm signal to be played through the speaker 29. There may be different alarm signals for different conditions For example, a low battery alarm can be distinctly different from the alarm signal for a heart attack.

7. Depressing the button 22 will turn off the alarm signal being played.

8. The patient should then follow his doctor's instructions as to what to do for the particular alarm that is generated. The instructions could be spoken by a recording within the PAD 20, displayed on a visual display on the PAD or simply listed on a label attached to the PAD 20.

It is envisioned that there might be a multiplicity of alerting or alarm signals generated by the alarm circuitry 27. For example there might be a minor alarm that will prompt the patient to schedule an appointment with their doctor and a major alarm where the patient should proceed immediately to obtain medical care. Examples of two such conditions are an irregular heartbeat that might require a change in patient medication as a minor alarm and the detection by the implant of an S-T segment shift indicating the onset of a heart attack being a major alarm.

FIG. 3 also shows a connector 26 to a, repeater module such as element 28 of FIG. 2. This would allow the patient to connect a telecommunications device to the PAD 20 to enable transmission of data from the implant 10 to remotely located external equipment and services.

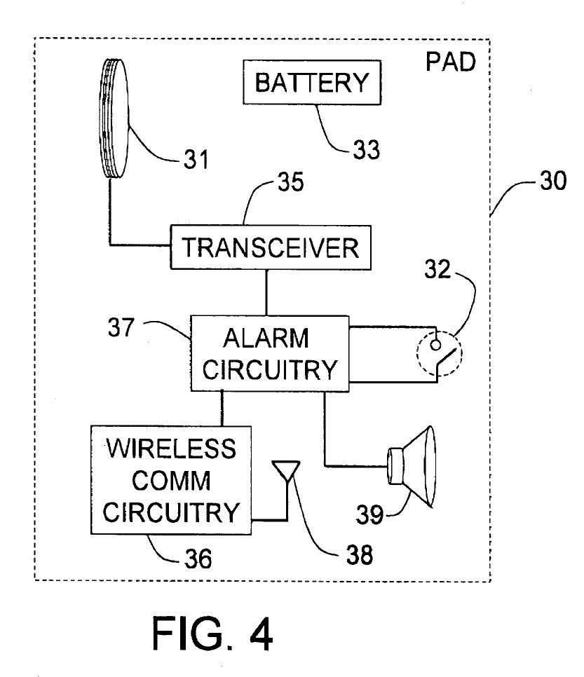

FIG. 4 is a block diagram of the elements within a repeater PAD 30 that has an integrated within the house or long-range telecommunications capability using the wireless communication circuitry 36 and antenna 38. As with the PAD 20 of FIG. 3, the repeater PAD 30 of FIG. 4 includes a battery 33 and a coil 31 connected to a transceiver 35. Alarm circuitry 37 connected to the speaker 39 can process incoming signals from the implant 10 and generate alarm signals to be emitted by the speaker 39. The button (switch) 32 can turn off the alarm signal and can be used as described with FIG. 1 to initiate data communication between the implant 10 and the PAD 30 or to check the status of either or both devices. The repeater PAD 30 can also transmit alert or alarm information via the wireless communications circuitry 36 and antenna 38 to remotely located external equipment and/or services that would facilitate rapid response to patient conditions. The external equipment might also include a programmer or display terminal that could process and display data from the implant 10 to help diagnose and treat the patient's condition.

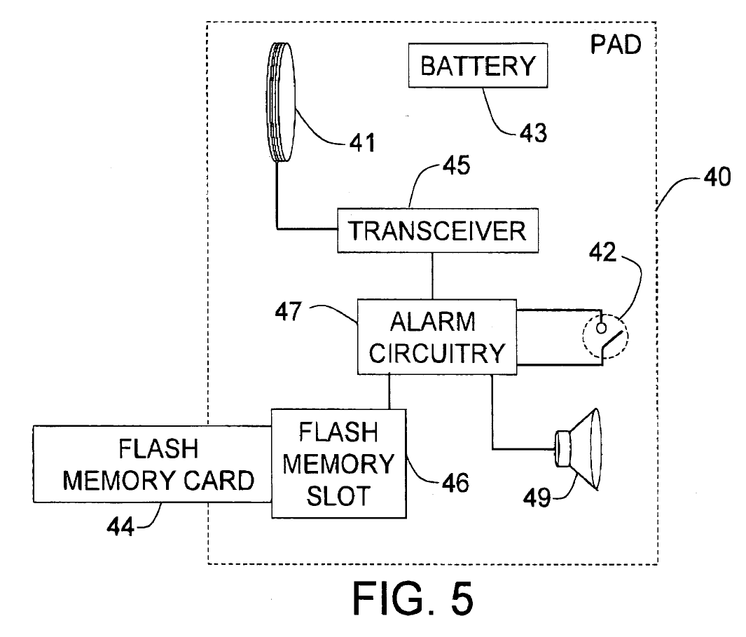

FIG. 5 is a block diagram of the elements within a PAD 40 with additional patient monitoring flash memory capability. As with the PAD 20 of FIG. 3, the PAD 40 of FIG. 4 includes a battery 43 and a coil 41 connected to a transceiver 45. Alarm circuitry 47 connected to the speaker 49 can process incoming signals from the implant 10 and generate alarm signals to be emitted by the speaker 49. The button (switch) 42 can turn off the alarm signal and can be used as described with FIG. 1 to initiate data communication between the implant 10 and the PAD 40 or to check the status of either or both devices. The PAD 40 can also store received data including alarm information in a flash memory card 44 inserted into the flash memory slot 46. In this way the PAD 40 in conjunction with the implant 10 could function as a Holter monitor system allowing outpatient collection of cardiac, neurological and/or other patient data. The flash memory card 44 could be either a proprietary interface or a standardized memory card. Examples of standardized flash memory cards are compact flash cards, PCMCIA flash cards, SD (Secure Digital) memory cards, Smart Media memory cards, USB flash memory adaptors, memory stick flash memory cards and multimedia memory cards. Thus, the PAD 50 could enhance the functionality of an implanted pacer of ICD to provide similar functionality as the Medtronic REVEAL without the need for an additional implant.

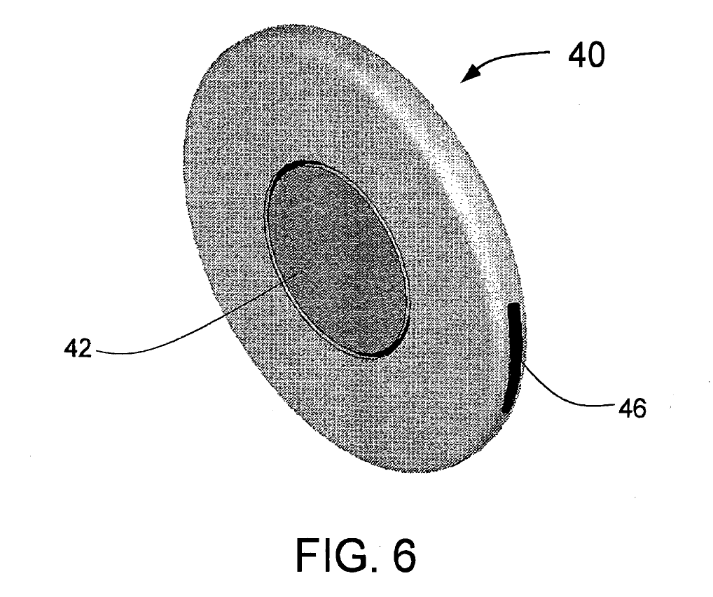

FIG. 6 illustrates the physical configuration of a PAD 40 with a flash memory slot 46 and button 42. The shell of the PAD 40 is typically made from a hard plastic that is molded to the appropriate size and shape.

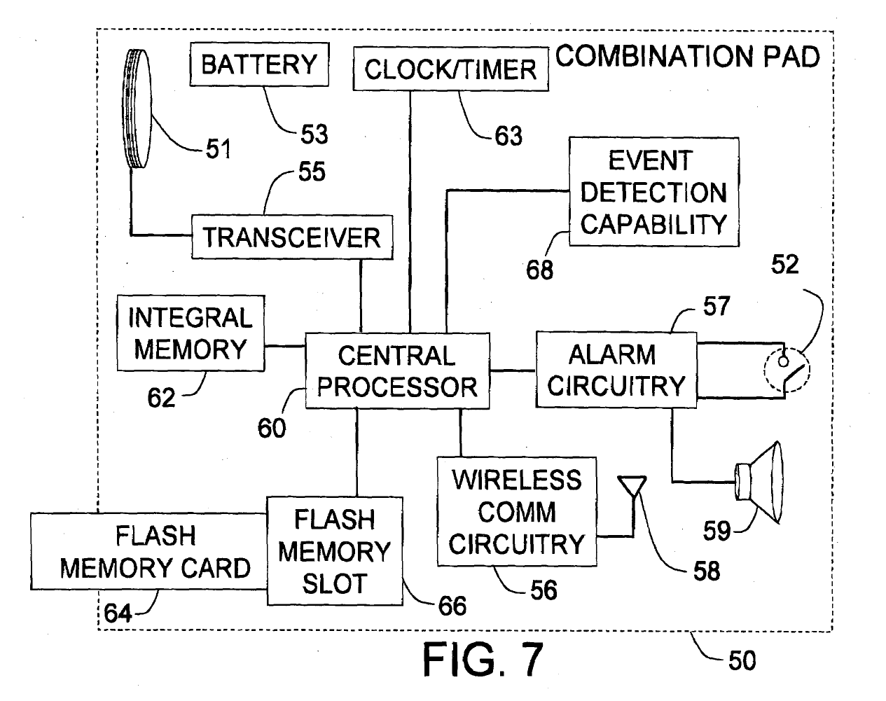

FIG. 7 is a block diagram of the elements within a combination PAD 50 powered by the battery 53 combining the capabilities of a basic alarm PAD, a repeater PAD and a memory PAD with additional event detection capability. A central processor 60 with integral memory 62 can send and receive data from an implanted device through the transceiver 55 and coil 51. The central processor 60 can also engage the event detection capability 68 to detect specific medical events by processing data received from the implant. The event detection capability 68 can be electronic circuitry (hardware), a software algorithm or a combination of hardware and software.

A clock/timer 63 can enhance the functionality of the PAD 50 by allowing the storage of the actual or relative time of received or detected events.

Alarm circuitry 57 connected to the speaker 59 can generate alarm signals to be emitted by the speaker 59. The button (switch) 52 can turn off the alarm signal and can be used as described with FIG. 1 to initiate data communication between the implant 10 and the PAD 50 or to check the status of either or both devices.

The combination PAD 50 can also transmit alert or alarm information via the wireless communications circuitry 56 and antenna 58 to remotely located external equipment and/or services that would facilitate rapid response to patient conditions. The external equipment might also include a programmer (not shown) or display terminal (not shown) that could process and display data from the implant to help diagnose and treat the patient's condition.

The combination PAD 50 can also store received data including alarm information in the integral memory 62 or a flash memory card 64 inserted into the flash memory slot 66. In this way the combination PAD 50 in conjunction with the implant could function as a Holter monitor system allowing outpatient collection of cardiac, neurological and/or other patient data. The flash memory card 64 could be either a proprietary interface or a standardized memory card. Examples of standardized flash memory cards are compact flash cards, PCMCIA flash cards, SD (Secure Digital) memory cards Smart Media memory cards, USB flash memory adaptors, memory stick flash memory cards and multimedia memory cards. Thus, the PAD 50 could enhance the functionality of an implanted pacer of ICD to provide similar (or superior) functionality as compared to the Medtronic REVEAL without the need for an additional implant.

Diagnostic information collected through any of the PAD configurations would allow the patient's doctor to prescribe appropriate therapies including drug regimens, cardiac catheter intervention, and/or electrical stimulation. Such therapies could also include modifications in the existing therapy provided by the implanted medical device from which the diagnostic data was collected.

It is important to understand how a patient could use the PAD for detecting a medical condition that could be life threatening or, at least, a medical condition that would require prompt attention. An example of a method that a patient could use with the PAD in conjunction with an implanted medical device is as follows:

A) the patient would have implanted a medical device such as a pacer or ICD that can detect a cardiac event such as an arrhythmia, an ST segment shift that is indicative of a heart attack or any other cardiac rhythm anomaly;

B) the patient would also have a patient alerting device (PAD) that can receive short-range telemetry from the implanted medical device; and

C) the implanted medical device and the PAD would be programmed that if a cardiac event is detected by the implanted medical device, the PAD would produce an alarm signal that the patient could use to promptly seek medical assistance.

The patient might have the PAD in place in close proximity to the implanted medical device on an almost fulltime basis. However, one important method for using this system would have the patient place the PAD over the site of the implanted medical device only when he or she senses that a cardiac event may be occurring. A specific example would be a patient who has an implanted pacer or ICD that has the capability to the send out an electrogram signal by short-range telemetry from one or more electrodes located in the patient's heart, through the implanted device and into the PAD. The PAD could then be programmed to detect the occurrence of a heart attack by the means and methods described in the Fischell et al patent application Ser. No. 10/251,505. This could include the comparison of the real-time electrogram with a baseline electrogram that was previously recorded in either the implanted device or in the PAD. Patients may not typically wish to wear the PAD all the time, but they would be readily willing to place the PAD over the site of the implanted device if they believed that they had some symptom of a cardiac event. Also patients are frequently in denial about the occurrence of the symptoms of a heart attack such as severe indigestion or chest pain, etc, because they do not wish to go to an emergency room. However, if a patient had an implanted device and a PAD and if he or she sensed some symptom that could be a serious cardiac event such as a heart attack, that patient could use the PAD to either relieve his or her anxiety or be directed by the PAD to immediately proceed to a medical facility for treatment. It should be understood that the detection of the cardiac event could be either in the implanted medical device or in the PAD.

If the cardiac event was troublesome but did not require immediate medical attention, e.g., premature ventricular or atrial contractions, then the PAD could provide a different alarm signal that would indicate that medical attention should be obtained but that the situation was not an emergency. Furthermore the repeater PAD could be used to access remotely located equipment and/or services that could be used to review the patient's electrogram and communicate with the patient (e.g., by cell phone) to provide medical advice and/or to call for an ambulance if an emergency situation was occurring.

Although much of the discussion herein has been related to detection of coronary events such as AMI, the PAD might be used with existing or future implanted devices with one or more of the following capabilities:

1. The ability to measure blood glucose and/or insulin levels;

2. The ability to measure blood pressure or blood oxygen levels;

3. The ability to detect the process EEG information;

4. The ability to measure body temperature;

5. The ability to measure liquid level in a patient's bladder (this could allow a patient actuated valve to be more effective for incontinent patients);

6. The ability to measure changes from the normal variation in the R-R interval of a patient's heart beat. It is well known that R-R interval varies over time with two frequencies of approximately 0.1 Hz and 0.3 Hz corresponding to the activity in the sympathetic and parasympathetic nervous systems. Changes in the amplitude of either the sympathetic or parasympathetic R-R interval variations can be indicative of a number of oncoming medical conditions.

In any of the above measurements, it is envisioned that detection of events could either be within the implanted medical device or by the PAD itself through processing of data the PAD receives from the implant.

Various other modifications, adaptations, and alternative designs are of course possible in light of the above teachings. Therefore, it should be understood at this time that, within the scope of the appended claims, the invention can be practiced otherwise than as specifically described herein

What is claimed is:

1. A patient alerting system including:
an implanted medical device having short-range telemetry and being capable of detecting one or more different detected events related to the medical condition of a patient in whom the implanted medical device is implanted; and
a patient alerting device situated in close proximity to the implanted medical device and being capable of receiving the short-range telemetry from the implanted medical device, the patient alerting device also having the capability to alert the patient by means of an alarm signal after the detected event is detected by the implanted medical device.

2. The system of claim 1 where the detected event that is detected by the implanted device is selected from the group consisting of bradycardia, ventricular fibrillation, a cardiac arrhythmia, acute myocardial ischemia, a heart attack, a precursor of an epileptic seizure, the aura of a migraine headache, low or high blood glucose, low or high blood pressure, change from a normal R-R interval variation of the electrocardiogram, or any similar medical condition.

3. The system of claim 1 where the alarm signal is selected from the group consisting of a sound, a vibration or a visual display.

4. The system of claim 1 where the patient alerting device is designed to indicate the state of the battery of either or both the implanted medical device or the patient alerting device.

5. The system of claim 1 where the patient alerting device is designed is record in memory data received from the implanted medical device.

6. The system of claim 1 further including a repeater module capable of transmitting data received by the patient alerting device to remotely located external equipment.

7. The system of claim 6 where the repeater module is attached to the patient alerting device.

8. The system of claim 6 where the repeater module is integrated into the patient alerting device.

9. The system of claim 1 where the patient alerting device further comprises an electrical switch capable of interacting with the circuitry in the patient alerting device.

10. The system of claim 9 where the switch is a button on the surface of the patient alerting device.

11. The system of claim 9 where the switch can turn off the alarm signal originating from the patient alerting device.

12. The system of claim 9 where the switch is designed to initiate communication between the patient alerting device and the implanted medical device.

13. The system of claim 9 where the switch can initiate a status check of the patient alerting device.

14. The system of claim 9 where the switch can initiate a check of the status of the operating characteristics of the implanted medical device by means of the short-range telemetry.

15. The system of claim 9 where the switch can initiate a status check of both the patient alerting device and the implanted medical device.

16. The system of claim 1 further including a flash card slot located in the patient alerting device, the flash card slot being designed to receive a flash memory card.

17. The system of claim 16 where the flash memory card is selected from the group consisting of compact flash cards, PCMCIA flash cards, SD (Secure Digital) memory cards, Smart Media memory cards, USB flash memory adaptors, memory stick flash memory cards and multimedia memory cards.

18. A repeater patient alerting device designed to extend the data communication range of an implanted medical device, the repeater patient alerting device having near-field communications capability to send and receive data from the implanted medical device and limited-range telemetry capability to allow the implanted medical device to communicate with external equipment within a distance of less than 100 meters.

19. The device of claim 18 where the limited-range data communication means uses a standardized protocol.

20. The device of claim 19 where the standardized protocol is selected from the group consisting of: Bluetooth, 802.11.a, 802.11.b or 802.11.g.

21. The device of claim 18 where the external equipment is selected from the group consisting of modems, printers, personal computers, laptop PCs, tablet PCs, cell phones, PDAs, paging devices, and implantable device programmers.

22. The device of claim 18 where the external equipment includes long-range communication means to facilitate data communication between the implanted medical device and remotely located systems and services at a distance of 100 meters or more from the implanted medical device.

23. A repeater patient alerting device designed to extend the data communication range of an implanted medical device, the repeater patient alerting device having near-field communications means to send and receive data from the implanted medical device and long-range data communication means to allow the implanted medical device to communicate with remotely located external equipment located at a distance of more than 100 meters from the implanted medical device.

24. The device of claim 23 where the long-range data communication means uses a standardized protocol.

25. The device of claim 24 where the standardized protocol is selected from the group consisting of CDMA, TDMA, GPRS and SMS.

26. A method for a patient to determine if he or she is having a cardiac event, the method including the following steps:
A) implant within the patient an implanted medical device having short-range telemetry and being capable of detecting one or more different detected events related to the medical condition of a patient in whom the implanted medical device is implanted; and
B) place a patient alerting device in close proximity to the implanted medical device, the patient alerting device being capable of receiving the short-range telemetry from the implanted medical device, the patient alerting device also having the capability to alert the patient by means of an alarm signal when the detected event is detected by the implanted medical device.

27. The method of claim 26 where the implanted medical device has the capability to detect at least one medical condition that is selected from the group consisting of bradycardia, ventricular fibrillation, a cardiac arrhythmia, acute myocardial ischemia, a heart attack, a precursor of an epileptic seizure, the aura of a migraine headache, low or high blood glucose, low or high blood pressure, change from a normal R-R interval variation of the electrocardiogram, or any similar medical condition.

28. A system for detecting if a patient has an anomaly of his or her heart rhythm, the system including:
an implanted medical device having short-range telemetry and being capable of detecting one or more different detected events related to the patient's heart rhythm; and
a patient alerting device situated in close proximity to the implanted medical device and being capable of receiving the short-range telemetry from the implanted medical device, the patient alerting device also having the capability to store in memory the electrogram and/or electrocardiogram signal that is detected by the implanted medical device and transmitted to the patient alerting device by the short-range telemetry.

29. The system of claim 28 where the patient alerting device includes a means for the patient to initiate recording of the electrogram or the electrocardiogram.

30. A system for detecting an event related to the medical condition of a human patient the system including:
an implanted medical device having short-range telemetry and being capable of measuring one or more different aspects of a patients medical condition;
a patient alerting device having processing capability situated in close proximity to the implanted medical device and being capable of receiving the short-range telemetry from the implanted medical device, the patient alerting device also having the capability to detect an event related to the medical condition of the patient by processing the telemetry received from the implanted medical device.

31. The system of claim 30 further including means to alert the patient following detection of the event related to the medical condition of the patient.

32. The system of claim 30 further including means to store the telemetry received by the patient alerting device from the implanted medical device.

33. The system of claim 30 further including means to store the time of occurrence of the detected event.

34. The system of claim 30 further including means to transmit data to external equipment following the occurrence of the detected event.
